(19) United States
(12) Patent Application Publication
(10) Pub. No.: US 2026/0306295 A1
(43) Pub. Date: Oct. 8, 2026

(54) OPHTHALMIC INJECTION ROBOT AND SYSTEM

(71) Applicant: Alcon Inc., Fribourg (CH)

(72) Inventor: Paul R. HALLEN, Colleyville, TX (US)

(21) Appl. No.: 19/456,643

(22) Filed: Jan. 22, 2026

Related U.S. Application Data
(60) Provisional application No. 63/751,501, filed on Jan. 30, 2025.

(51) Int. Cl. A61F 9/00; A61B 3/10; A61B 3/12; A61B 17/00; A61B 17/34; A61B 34/10; A61B 34/20; A61B 34/30; A61B 34/35; A61B 90/00; A61B 90/35; G16H 20/17; G16H 40/67

(52) U.S. Cl. CPC A61F 9/0017; A61B 3/1005; A61B 3/102; A61B 3/1225; A61B 34/35; A61B 90/35; G16H 20/17; G16H 40/67; A61B 2017/00477; A61B 2017/3409; A61B 2034/107; A61B 2034/2055; A61B 2034/2065; A61B 2034/302; A61B 2090/373; A61B 2505/05

(57) ABSTRACT

In certain embodiments, an ophthalmic injection robot includes a frame, a robotic arm, a docking assembly, and a control system. The frame includes a patient support and positioning cameras. The docking assembly is coupled to the robotic arm, and includes fine adjustment cameras, and a staging assembly including fine adjustment actuators, a needle extension actuator, and a needle assembly including a needle. The control system includes a memory and a processor configured to receive the fine adjustment image data generated by fine adjustment cameras, process the fine adjustment image data to generate a blood vessel map of an eye of a patient, select an injection site on the eye of the patient based on the blood vessel map, and control the fine adjustment actuators to position the needle above the selected injection site for target-confirmed injection of a drug through the needle and into the eye of the patient.

### CROSS-REFERENCE TO RELATED APPLICATIONS

This application claims the benefit of U.S. Provisional Application Ser. No. 63/751,501 (filed on Jan. 30, 2025), the content of which is incorporated by reference herein in its entirety.

### BACKGROUND

The present disclosure relates to a robotic system, and, more particularly, to a robotic system for an ophthalmic injection facility.

An ophthalmic injection delivers medication directly into the eye of a patient using a needle. The structures of the eye are extremely small and delicate and, therefore, ophthalmic injections are performed by skilled ophthalmic surgeons. Becoming an ophthalmic surgeon requires many years of training, and the time spent by the ophthalmic surgeon in the treatment room of an ophthalmic injection facility is therefore a very valuable resource.

### SUMMARY

In certain embodiments, an ophthalmic injection system includes an ophthalmic injection robot. The ophthalmic injection robot includes a frame, a robotic arm, a docking assembly, and a control system. The frame includes a patient support and positioning cameras configured to generate positioning image data. The docking assembly is coupled to the robotic arm, and includes fine adjustment cameras and a staging assembly. The fine adjustment cameras are configured to generate fine adjustment image data. The staging assembly includes fine adjustment actuators, a needle extension actuator, and a needle assembly with a needle. The control system includes a memory and a processor in data communication with the memory. The processor is configured to receive the fine adjustment image data generated by fine adjustment cameras, process the fine adjustment image data to generate a blood vessel map of an eye of a patient, select an injection site on the eye of the patient based on the blood vessel map, and control the fine adjustment actuators to position the needle above the selected injection site for target-confirmed injection of a drug through the needle and into the eye of the patient.

In certain embodiments, the ophthalmic injection system also includes a visualization computer coupled to the ophthalmic injection robot over a network. The visualization computer includes a display, an input device, a memory, and a processor configured to present, on the display, the blood vessel map and the selected injection site, responsive to a user input, generate the injection site approval command or the injection site disapproval command, send the injection site approval command or the injection site disapproval command to the control system of the ophthalmic injection robot.

### BRIEF DESCRIPTION OF THE DRAWINGS

FIG. 1 depicts an ophthalmic injection robot, in accordance with embodiments of the present disclosure.

FIG. 2A depicts a top view of a docking assembly of the ophthalmic injection robot, in accordance with embodiments of the present disclosure.

FIG. 2B depicts a partial side view of the docking assembly depicted in FIG. 2A, in accordance with embodiments of the present disclosure.

FIG. 3 depicts a block diagram of the ophthalmic injection robot depicted in FIG. 1, in accordance with embodiments of the present disclosure.

FIG. 4A depicts a block diagram of an ophthalmic injection system, in accordance with embodiments of the present disclosure.

FIG. 4B depicts a block diagram of a visualization computer, in accordance with embodiments of the present disclosure.

FIG. 5 presents a sectional view of an eye depicting several types of ophthalmic injections.

FIG. 6 depicts a view of an eye with several candidate injection sites for an intravitreal injection, in accordance with embodiments of the present disclosure.

### DETAILED DESCRIPTION

Ophthalmic injections (such as intravitreal injections, etc.) may be used to treat certain conditions, such as age-related macular degeneration (AMD), diabetic eye disease, retinal vein occlusion, etc. While the number of patients that receive monthly ophthalmic injections has been increasing, there are not enough skilled ophthalmic surgeons and ophthalmic injection facilities to keep up with this growing demand.

An ophthalmic injection robot may improve patient throughput in ophthalmic injection facilities. However, intravitreal injections have complications if not performed with precision and care. One example is the complication of bleeding as a result of the injecting through a scleral or conjunctival capillary. At the pars plana (3 mm posterior from the limbus) the needle is pressed against the conjunctiva and penetrates through to the sclera and finally into the vitreous and the drug is injected. If not careful, the ophthalmic injection robot may penetrate through a capillary and cause bleeding, such as the larger capillaries on the sclera. Penetration of a capillary and the resultant bleeding is not preferred and is to be avoided.

Embodiments of the present disclosure advantageously provide a ophthalmic injection system that improves patient safety and reduces or minimizes mistakes. In certain embodiments, the ophthalmic injection system comprises an ophthalmic injection robot that includes, inter alia, a frame, a robotic arm, a docking assembly, and a control system. The control system includes a processor that is configured to receive fine adjustment image data generated by fine adjustment cameras, process the fine adjustment image data to generate a blood vessel map of an eye of a patient, select an injection site on the eye of the patient based on the blood vessel map, and control fine adjustment actuators to position a needle above the selected injection site for target-confirmed injection of a drug through the needle and into the eye of the patient.

The blood vessel map advantageously allows the processor of the ophthalmic injection robot to identify and avoid injection sites with capillaries and prevent bleeding.

After the needle is positioned above the selected injection site, the needle is extended onto the selected injection site (also known as the target), and the fine adjustment image data is processed to confirm that the needle is located on the selected injection site. After the needle location on the selected injection site is confirmed, the needle is extended into the eye of the patient, the drug is injected, and the needle is withdrawn. This process may be known as target-confirmed injection. If the needle is not confirmed to be located on the selected injection site, the needle may be repositioned using the fine adjustment actuators. In certain embodiments, the selected injection site may have a diameter within which the needle is to be located, which may be determined based on the blood vessel map. For example, the selected injection site diameter may be larger than the bore of the needle, which allows for a more relaxed placement of the needle. In another example, the selected injection site diameter may be smaller than the bore of the needle, which requires a more precise placement of the needle.

Advantageously, the docking assembly of the ophthalmic injection robot may include adjustable, high-contrast red green blue (RGB) light sources. The docking assembly may also include adjustable multispectral light sources, and the fine adjustment image data may also include multispectral image data when multispectral light sources are employed.

In certain other embodiments, the ophthalmic injection system also includes a visualization computer coupled to the ophthalmic injection robot over a network. The visualization computer includes a display, an input device, a memory, and a processor configured to present, on the display, the blood vessel map and the selected injection site, responsive to a user input, generate the injection site approval command or the injection site disapproval command, send the injection site approval command or the injection site disapproval command to the control system of the ophthalmic injection robot.

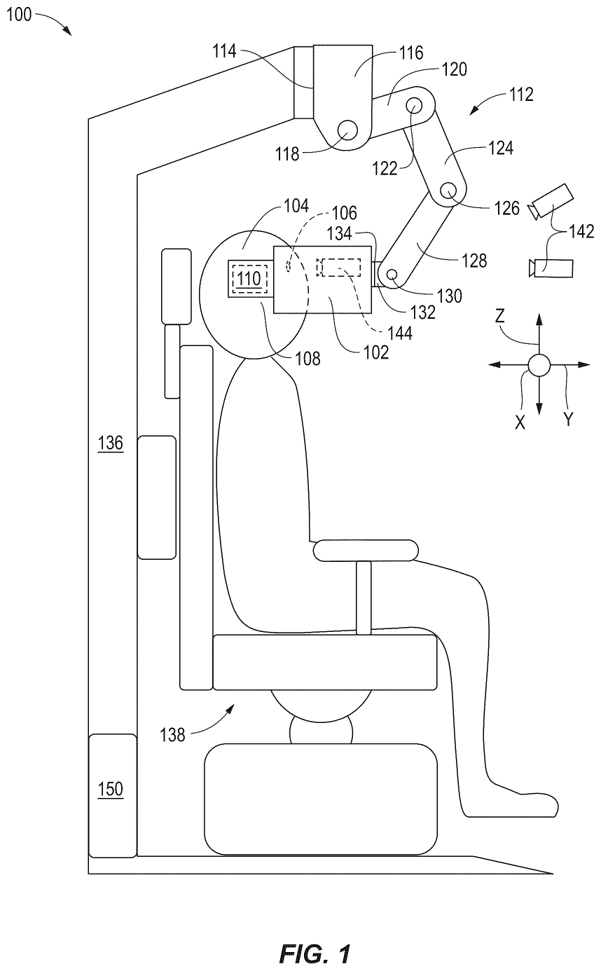

FIG. 1 depicts an ophthalmic injection robot 100, in accordance with embodiments of the present disclosure.

In certain embodiments, the ophthalmic injection robot 100 includes a docking assembly 102 that receives a portion of the head 104 of a patient and covers one or both eyes 106 of the patient. The docking assembly 102 may include some or all of a forehead rest, cheek rests, temporal rests, chin rest, or rests for engaging other portions of the head 104 of the patient, some or all of which may be adjustable. The docking assembly 102 may include structures for clamping or otherwise retaining the head 104 of the patient, such as clamping actuators 108 for pressing pads 110 against the head 104 of the patient, a headband encircling the head 104 of the patient and fastened to the docking assembly 102, or other structures.

The docking assembly 102 includes a robotic arm 112 for moving the docking assembly 102 into alignment with the head 104 of patients of various sizes. The robotic arm 112 may be understood with respect to X, Y, and Z direction, where the Z direction is substantially (e.g., within 2 degrees of) parallel to the direction of gravity and the X and Y directions are substantially (e.g., within 2 degrees of) perpendicular to the Z direction and to one another. The robotic arm 112 is configured to move the docking assembly 102 in the X, Y, and Z directions as well as one or more rotational degrees of freedom, such as rotation about an axis substantially (e.g., within 2 degrees of) parallel to the X, Y, and/or Z direction.

The robotic arm 112 may include a rotational joint 114 coupled to a base 116. The base 116 is coupled by elbow joint 118 to a link 120. Elbow joint 122 couples link 120 to link 124. Elbow joint 126 couples link 124 to link 128. Elbow joint 130 couples link 128 to link 132. Link 132 may be coupled by rotational joint 134 to the docking assembly 102. Each of the illustrated joints 114, 118, 122, 126, 130, 134 has a corresponding actuator for inducing movement of the joint. The robotic arm 112 may have at least five (5) degrees of freedom (DOF). For example, the illustrated robotic arm 112 has six (6) DOF. The robotic arm 112 may be embodied as a commercially available serial robotic arm. The robotic arm 112 may also be implemented as linear actuators, such as linear actuators implementing movements in the X, Y, and Z directions as well as one or more rotational actuators inducing rotation about one or more of the X, Y, and Z axes. For example, the robotic arm 112 may be embodied as gantry. Note that the illustrated size of the may be somewhat exaggerated relative to the size of the head 104 of the patient and may have a smaller relative size. For example, the robotic arm 112 may move the docking assembly 102 within a three-dimensional range of motion having dimensions in the X, Y, and Z directions that are less than 30 centimeters, 15 centimeters, or 10 centimeters.

The robotic arm 112 may be mounted to a support 136, such as by the rotational joint 114 mounting the illustrated robotic arm to the support 136. The support 136 may be mounted to a floor, wall, ceiling, movable cart, or other structure.

The robotic arm 112 may be mounted to a support 136, such as by the rotational joint 114 mounting the illustrated robotic arm to the support 136. The support 136 may be mounted to a floor, wall, ceiling, movable cart, or other structure. A patient support 138, such as a seat, etc., may be mounted to the support 136 or secured to a floor or wall adjacent the support 136. A harness may secure the patient to the patient support 138 to reduce movement of the head 104 of the patient relative to the docking assembly 102. The docking assembly 102, support 136, or other structure, may have speakers mounted thereto to which a medical professional can wirelessly connect and output verbal instructions or reassurance to the patient.

The docking assembly 102 and actuators of the robotic arm 112 may be coupled to a control system 150. The control system 150 may be housed within the support 136 or elsewhere. The control system 150 may receive positioning image data generated by the positioning cameras 142 in order to estimate a three-dimensional position of the head 104 of the patient and activate the robotic arm 112 to position the docking assembly 102 at or within a threshold distance of the head 104 of the patient. The docking assembly 102 itself may include fine adjustment cameras 144 that are configured to generate fine adjustment image data, which may be used by the control system 150 to perform fine adjustments to the position of the docking assembly 102. Alternatively, the docking assembly 102 may incorporate actuators that are controlled to perform fine adjustments of the docking assembly 102 based on the fine adjustment image data from the fine adjustment cameras 144.

The position of the docking assembly 102 itself may be determined by sensing a kinematic state of the robotic arm 112 using sensors incorporated into the joints 114, 118, 122, 126, 130, 134 or elsewhere in the robotic arm 112. Additionally, the position of the docking assembly 102 may also be determined based on positioning image data generated by the positioning cameras 142 and fine adjustment image data generated by the fine adjustment cameras 144.

Although the positioning cameras 142 and the fine adjustment cameras 144 are described as being used to estimate the position of the head 104 of the patient and possibly the docking assembly 102, other imaging or sensing modalities may be used such as light detection and ranging (LIDAR), radio detection and ranging (RADAR), ultrasonic sensing, or other type of sensor. The fine adjustment cameras 144 may each be replaced with an optical coherence tomography (OCT) device, scanning laser ophthalmoscope, or other type of imaging device. An OCT device is particularly helpful for tracking the location of a needle during insertion, injection, and withdrawal.

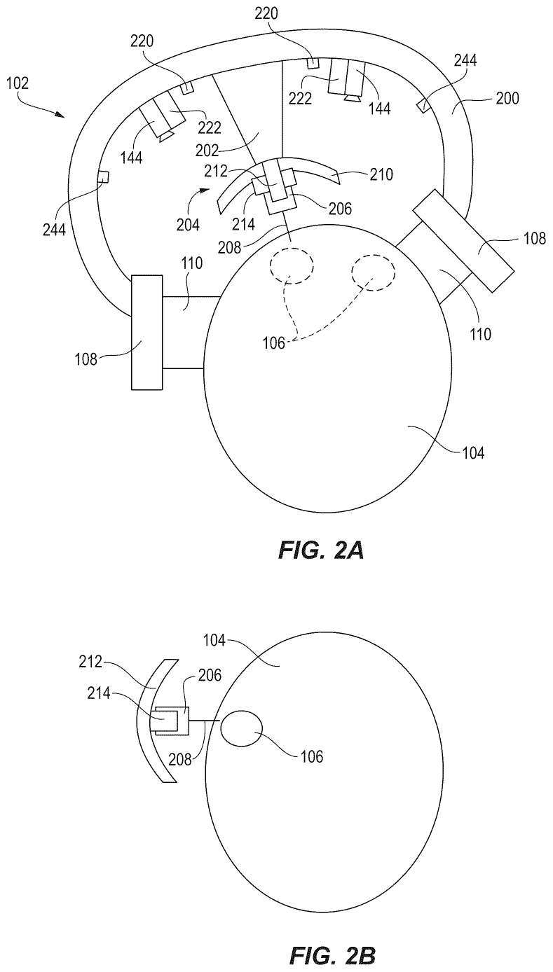

FIG. 2A depicts a top view of a docking assembly 102 of the ophthalmic injection robot 100, while FIG. 2B depicts a partial side view of the docking assembly depicted in FIG. 2A, in accordance with embodiments of the present disclosure.

In certain embodiments, the docking assembly 102 may include a frame 200. The frame 200 may be embodied as a track, rail, or other structural member along which components may be fastened at various positions. The frame 200 may include mounting structures 202 mounted thereto. Each mounting structure 202 has a staging assembly 204 mounted thereto. The staging assembly 204 includes fine adjustments actuators that perform fine adjustments, relative to the precision of the robotic arm 112, of the position of the needle assembly 206 for inserting a needle 208 of the needle assembly 206 into the eye 106 of the patient in order to inject the ophthalmic drug.

In the illustrated embodiment, the staging assembly 204 includes a fine adjustment actuator 210 and a fine adjustment actuator 212 that are oriented substantially (e.g., within 2 degrees of) perpendicular to one another. The fine adjustment actuators 210, 212 may be linear actuators or the illustrated fine adjustment actuators 210, 212. For example, the fine adjustment actuators 210, 212 may define arcuate actuation paths that are each centered on a remote center of motion. For example, the remote center of motion may lie on the needle 208 or a path followed by the needle 208 when extended by a needle extension actuator 214, which is a linear actuator configured to extend and withdraw the needle 208 when performing injections. For example, the fine adjustment actuator 210 may be mounted to the mounting structure 202, the fine adjustment actuator 212 may be mounted to the fine adjustment actuator 210 and be actuated thereby along a first actuate path. The needle extension actuator 214 may be mounted to the actuator and may be actuated thereby along a second arcuate path that has the same remote center of motion as the first arcuate path, e.g., within 1 mm, 0.01 mm, or 1 micron. The needle assembly 206 may be mounted to the needle extension actuator 214 with the needle 208, or a line extending along the center of the lumen of the needle 208 lying on the remote center of motion, e.g., within 1 mm, 0.01 mm, or 1 micron.

The clamping actuators 108 may be mounted to the frame 200. The clamping actuators 108 are configured to extend pads 110 into engagement with the head 104 of the patient in order to reduce movement of the head 104 relative to the docking assembly 102. For example, there may be one pad 110 coupled to each actuator 108 for comfortably clamping the head 104 of the patient.

Generally, the docking assembly 102 may be positioned relative to the head 104 of the patient using the robotic arm 112 and positioning image data from the positioning cameras 142. Fine adjustment image data generated by the fine adjustment cameras 144 may be used to determine the relative position of the eye 106 of the patient and perform fine adjustments using the robotic arm 112 based on the position. Once in position, the clamping actuators 108 may be activated to bring the pads 110 into engagement with the head 104 of the patient. Note that the position of the pads 110 may be asymmetric relative to the head 104 of the patient since the same docking assembly 102 may be used in two different positions to perform injections on the right and left eyes 106 of the patient. The actuation of the clamping actuators 108 may be guided by fine adjustment image data generated by the fine adjustment cameras 144. For example, the clamping actuators 108 may be used to adjust the relative positions of the docking assembly 102 and the head 104 of the patient.

The docking assembly 102 may position the needle 208 on a line that intersects a point on the eye 106 of the patient at a prescribed position and angle, or within a tolerance of such a position and angle that is within the range of motion provided by the staging assembly 204. For example, when performing intravitreal injection, the prescribed position may be between 3 and 3.5 millimeters from the limbus for an aphakic eye and between 3.5 and 4 millimeters from the limbus for a phakic eye. The prescribed angle may be selected such that upon insertion of the needle, the needle avoids contact with the lens and retina while placing medication near the retina or area of the retina to be treated.

The docking assembly 102 may include electronic components in addition to the fine adjustment cameras 144. The docking assembly 102 may include fixation device 220. Each fixation device 220 may be embodied as a static image, light source, screen for displaying a fixation device, or other device. A separate fixation device 220 may be provided for each eye 106 or a single fixation device 220 may be used for both right and left eyes 106. Alternatively, a single fixation device 220 may be mounted at different positions on the frame 200 for different eyes 106. In some embodiments a single fixation device 220 may be centrally located to be used for both eyes 106, so that each eye 106 may be directed toward the nose of the patient in order to expose the sclera for receiving an injection. Alternatively, a single screen implementing the fixation device 220 may display a fixation device at a different location for each eye 106. The location of the fixation device 220 may be adjusted using software executed by the control system 150 or by the ophthalmic surgeon in order to induce the patient to position the eye 106 at a desired angle.

The docking assembly 102 may include an intraocular pressure (IOP) sensors 222. The IOP sensors 222 may be a contact or non-contact sensor and may be used during intravitreal injection to ensure that the IOP of the eye 106 of the patient does not increase to unsafe levels. There may be separate IOP sensors 222 for each eye or a single IOP sensor 222 may be mounted at different positions on the frame 200 in order to measure the IOP of each eye 106.

The docking assembly 102 may include light sources 244 for the fine adjustment cameras 144, such as adjustable high-contrast RGB light sources, multispectral light sources, a combination of adjustable high-contrast RGB and multispectral light sources, etc.

In some embodiments, a speculum may be provided for the patient. The speculum engages the eyelid of the patient and withdraws the eyelid to reduce risk of interference with injection of the needle 208. The speculum may incorporate a force/torque sensor coupled to the control system 150, which uses the output of the force/torque sensor to limit the amount of pressure applied to the eye 106 and eyelid of the patient. The speculum may be statically mounted and engage the eyelid of the patient as the docking assembly 102 is brought into alignment with the eye 106 of the patient. Alternatively, the speculum may include an extending actuator that extends the speculum following alignment using one or both of the robotic arm 112 and the staging assembly 204.

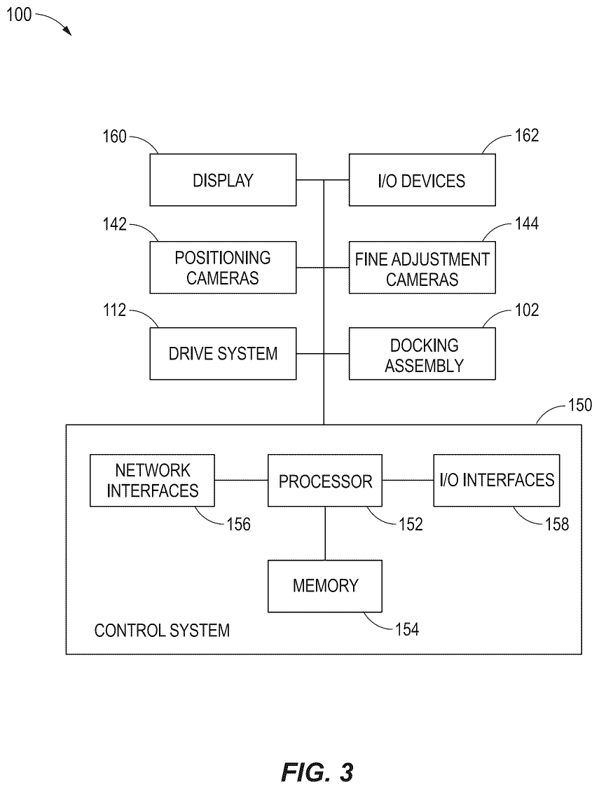

FIG. 3 depicts a block diagram of the ophthalmic injection robot 100 depicted in FIG. 1, in accordance with embodiments of the present disclosure.

The control system 150 is coupled to the robotic arm 112, the docking assembly 102, the positioning cameras 142, and the fine adjustment cameras 144. In certain embodiments, the control system 150 may include a processor 152 coupled to a memory 154, network interfaces 156, and input/output (I/O) interfaces 158. In some embodiments, the control system 150 may be coupled to one or more displays 160 (such as an ultra high definition (UHD) monitor, etc.), and one or more I/O devices 162 (such as a keyboard, mouse, trackpad, touchscreen monitor, etc.).

Generally, the memory 154 may store instructions that are executable by the processor 152, and the instructions, when executed, cause the processor 152 to perform various functions, such as receiving image data, processing image data, generating map data, controlling actuators, receiving user input, etc.

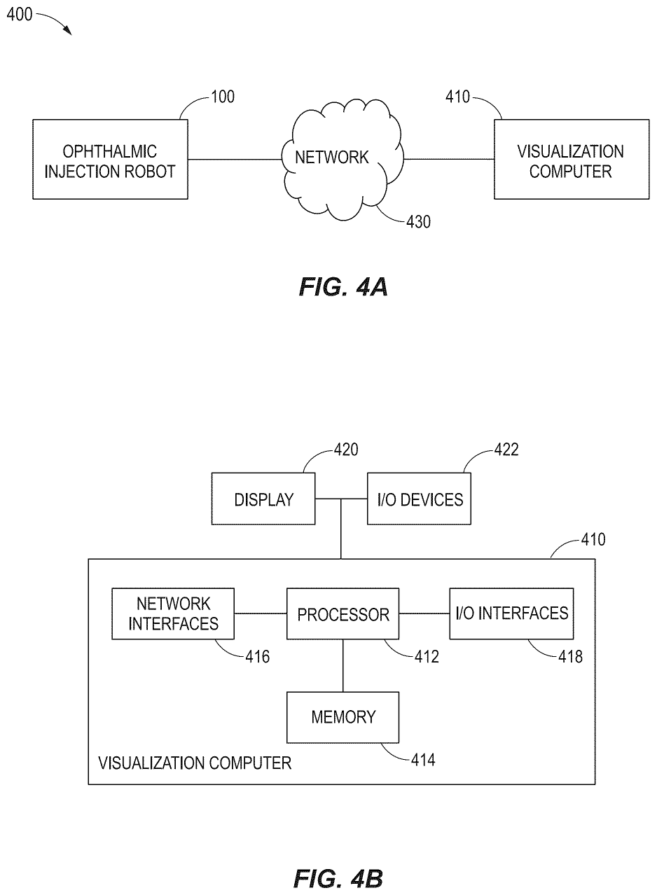

FIG. 4A depicts a block diagram of an ophthalmic injection system 400, in accordance with embodiments of the present disclosure.

In certain embodiments, the ophthalmic injection system 400 includes the ophthalmic injection robot 100 and a visualization computer 410. The ophthalmic injection robot 100 and the visualization computer 410 are in data communication over the network 430.

FIG. 4B depicts a block diagram of a visualization computer 410, in accordance with embodiments of the present disclosure.

In certain embodiments, the visualization computer 410 may include a processor 412 coupled to a memory 414, network interfaces 416, and I/O interfaces 418. The visualization computer 410 may be coupled to one or more displays 420 (such as an ultra high definition (UHD) monitor, etc.), and one or more I/O devices 422 (such as a keyboard, mouse, trackpad, touchscreen monitor, etc.).

Generally, the memory 414 may store instructions that are executable by the processor 412. The instructions, when executed, cause the processor 412 to perform various functions, such as receiving map data, processing map data, receiving user input, etc.

Exemplary processor-based methods for performing an ophthalmic injection using the ophthalmic injection robot 100 of the ophthalmic injection system 400 will now be discussed.

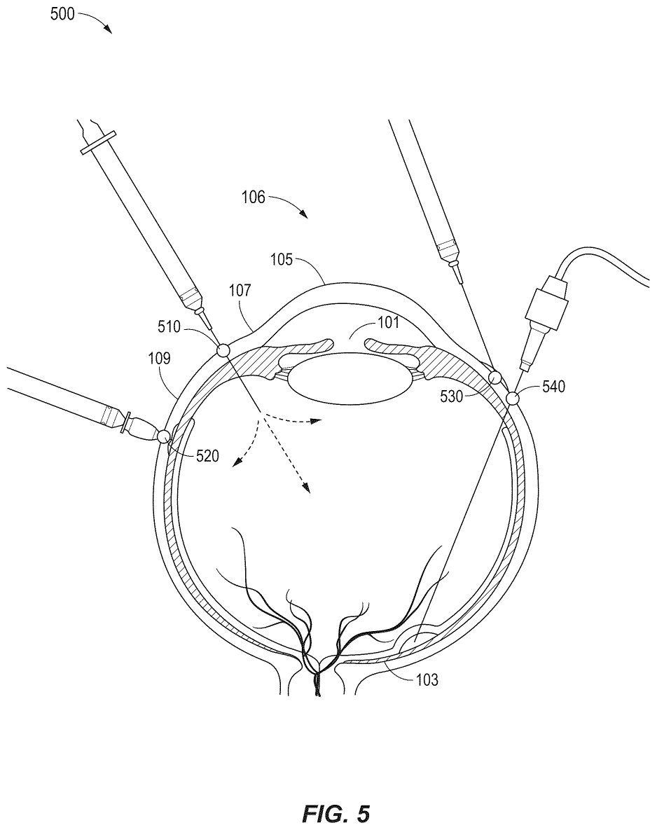

FIG. 5 presents a sectional view 500 of the eye 106 depicting several types of ophthalmic injections, including an intravitreal injection 510, a suprachoroidal injection 520, a subconjunctival injection 530, and a subretinal injection 540. The pupil 101, the retina 103, the cornea 105, the limbus 107, and the sclera 109 are also identified. An intravitreal injection 510 will be used to illustrate certain aspects of the ophthalmic injection system 400.

In certain embodiments, the patient is first seated (and secured) in the patient support 138. In response to receiving a start command, the processor 152 of the ophthalmic injection robot 100 sends a command to the positioning cameras 142 to begin generating positioning image data. The start command may be generated by the processor 152 in response to receiving user input from an ophthalmic surgeon, an attending nurse, a scrub technician, etc. using an I/O device 162. Alternatively, the start command may be generated by the processor 412 of the visualization computer 410 in response to receiving user input from an ophthalmic surgeon, an attending nurse, a scrub technician, etc. using an I/O device 422, and then sent over the network 430 to the ophthalmic injection robot 100.

The processor 152 then receives the positioning image data generated by the positioning cameras 142, and controls the docking assembly 102 to couple the docking assembly 102 to the head 104 of the patient based on the positioning image data. After the docking assembly 102 is coupled to the head 104 of the patient, the processor 152 sends a command to the fine adjustment cameras 144 to begin generating fine adjustment image data. In some embodiments, the fine adjustment cameras 144 may begin generating fine adjustment image data in response to the start command.

The processor 152 then receives the fine adjustment image data generated by fine adjustment cameras 144, and processes the fine adjustment image data to generate a blood vessel map of the eye 106 of the patient. One or more light sources 244, such as high-contrast RGB light sources, multispectral light sources, etc., may be adjusted to enhance the fine adjustment image data generated by the fine adjustment cameras 144.

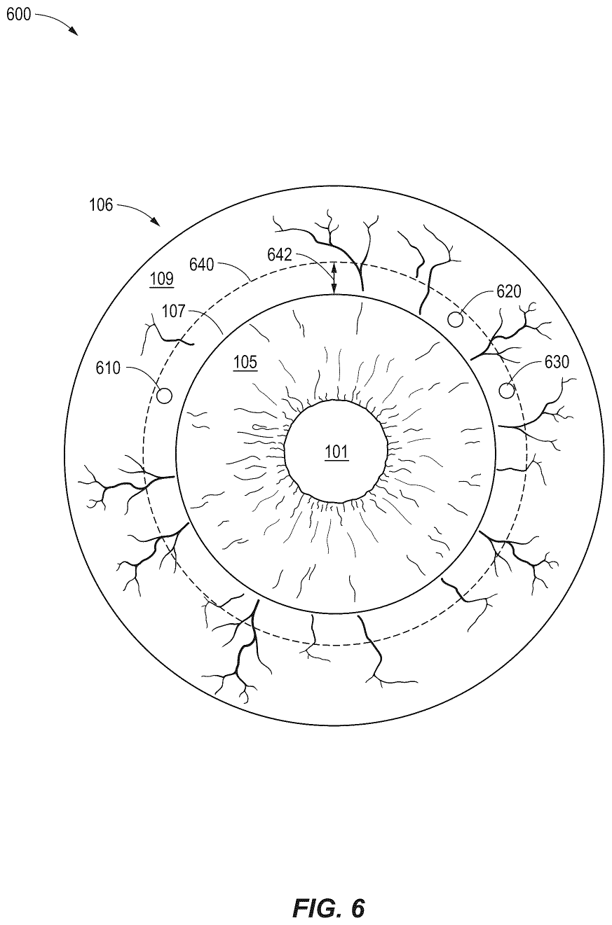

In certain embodiments, the blood vessel map may be a two-dimensional (2D) representation of certain structures of the eye 106, such as the cornea 105, the limbus 107, and the sclera 109 (see FIGS. 5, 6), and the blood vessels of the eye 106, such as the capillaries of the limbus 107 and sclera 109. Each structure in the blood vessel map may be represented by a series of X, Y coordinates that define its boundaries or location. Generally, the processor 152 may identify candidate injection sites that avoid the capillaries of the limbus 107 and the sclera 109 based on the blood vessel map.

In certain other embodiments, the blood vessel map may be a three-dimensional (3D) representation of certain structures of the eye 106, such as the cornea 105, the limbus 107, the sclera 109, and the retina 103. Each structure in the blood vessel map may be represented by a series of X, Y, Z coordinates that define its boundaries or location. A 3D blood vessel map may also include the arteries and veins of the eye 106, which also need to be avoided for certain types of ophthalmic injections. Generally, the processor 152 may identify candidate injection sites that avoid the capillaries of the limbus 107 and the sclera 109, as well as the arteries and veins of the eye 106, based on the blood vessel map.

The processor 152 then selects an injection site on the eye 106 based on the blood vessel map.

In certain embodiments, the processor 152 may identify a number of candidate injection sites based on the injection type and the blood vessel map. For example, for an intravitreal injection 510, the blood vessel map may be used to identify candidate injection sites that are located proximate to the limbus 107, such as within about 2 mm, 3 mm, 4 mm, etc., of the limbus 107. In certain embodiments, the processor 152 may determine a location and a score for each candidate injection site. The location may be the X, Y coordinates or the X, Y, Z coordinates of the candidate injection site in the blood vessel map. The score may represent the quality, desirability, etc., of the candidate injection site, and may be determined based on, inter alia, the distances from the candidate injection site location to the nearest capillaries.

FIG. 6 depicts a view 600 of the eye 106 with several candidate injection sites for an intravitreal injection, in accordance with embodiments of the present disclosure. The pupil 101, the cornea 105, the limbus 107, and the sclera 109 are identified, as well as a guide ring 640 that is centered on the pupil 101 and located a predetermined distance 642 of about 3 mm±1 mm from the limbus 107.

Three example candidate injection sites are depicted. The candidate injection site 610 may have a high score because the capillaries are located far from its location. The candidate injection site 620 may have a middle score because the capillaries are located nearer to its location. The candidate injection site 630 may have a low score because the capillaries are located close to its location.

The processor 152 may then select the injection site based on the candidate injection site scores (which are based on the blood vessel map). In the example depicted in FIG. 6, the processor 152 may select the candidate injection site 610 due to its high score.

In certain embodiments, the processor 152 may determine the candidate injection site locations based on previous patient preference data acquired from one or more prior injections. The patient preference data may include feedback from the patient regarding previous injection site locations and any associated pain, discomfort, adverse effects, etc. The patient preference data may be stored in memory 154 of the ophthalmic injection robot 100. For example, three candidate injection sites may be identified based on patient preference data. In some embodiments, the processor 152 may determine the scores for the patient-preferred candidate injection sites based on the blood vessel map, and then select the patient-preferred candidate injection site with the highest score.

In certain embodiments, the processor 152 may be configured to execute one or more machine learning (ML) models to process the fine adjustment image data, identify the candidate injection sites, and select the injection site.

After the injection site is selected, the processor 152 may receive an injection site approval command or an injection site disapproval command. Generally, in response to receiving the injection site approval command, the processor 152 may control the fine adjustment actuators 210, 212, the needle extension actuator 214, and the needle 208 to deliver the drug into the eye 106 of the patient. And, in response to receiving the injection site disapproval command, the processor 152 may select a different candidate injection site for further approval.

In certain embodiments, the processor 152 of the ophthalmic injection robot 100 may present, on the display 160, the blood vessel map and the selected injection site, and, in response to a user input from an I/O device 162, generate the injection site approval command or the injection site disapproval command.

In certain other embodiments, the processor 412 of the visualization computer 410 may receive the blood vessel map and the selected injection site from the ophthalmic injection robot 100 over the network 430, and present the blood vessel map and the selected injection site on the display 420. In some embodiments, the processor 412 may superpose the guide ring 640 on the blood vessel map at the predetermined distance 642, and then present the guide ring 640, the blood vessel map, the candidate injection sites 610, 620, 630, and the selected injection site 610 on the display 420. Responsive to a user input from an I/O device 422, the processor 412 may generate the injection site approval command or the injection site disapproval command, and send the injection site approval command or the injection site disapproval command to the ophthalmic injection robot 100 over the network 430.

After receiving the injection site approval command, the processor 152 then controls the fine adjustment actuators 210, 212 to position the needle 208 above the selected injection site for target-confirmed injection of a drug through the needle 208 and into the eye 106 of the patient.

In certain embodiments, after the needle 208 is positioned above the selected injection site, the processor 152 controls the needle extension actuator 214 to extend the needle 208 onto the selected injection site (or target), and the fine adjustment image data is processed to confirm that the needle 208 is located on the selected injection site. After the needle location on the selected injection site is confirmed, the processor 152 controls the needle extension actuator 214 to extend the needle 208 into the eye 106 of the patient, controls the needle 208 to inject the drug, and then controls the needle extension actuator 214 to withdraw the needle 208 from the eye 106 of the patient. After the needle 208 has been withdrawn from the eye 106 of the patient, the processor 152 controls the docking assembly 102 to decouple the docking assembly 102 from the head 104 of the patient based on the positioning image data.

If the needle 208 is not confirmed to be located on the selected injection site, the processor 152 may reposition the needle 208 using the fine adjustment actuators 210, 212. In certain embodiments, the selected injection site may have a diameter within which the needle 208 is to be located, which may be determined based on the blood vessel map. For example, the selected injection site diameter may be larger than the bore of the needle 208, which allows for a more relaxed placement of the needle 208. In another example, the selected injection site diameter may be smaller than the bore of the needle 208, which requires a more precise placement of the needle.

For a subretinal injection 540, in certain embodiments, the ophthalmic injection robot 100 may include an OCT sensor that is configured to generate multidimensional retina image data. The processor 152 may determine a retina thickness based on the multidimensional retina image data, and further identify the candidate injection sites based on the retina thickness.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. An ophthalmic injection system, comprising:
an ophthalmic injection robot comprising:
a frame comprising a patient support, and positioning cameras configured to generate positioning image data;
a robotic arm;
a docking assembly coupled to the robotic arm, the docking assembly comprising:
fine adjustment cameras configured to generate fine adjustment image data, and
a staging assembly including fine adjustment actuators, a needle extension actuator, and a needle assembly including a needle; and
a control system comprising:
a memory, and
a processor in data communication with the memory, the processor configured to:
receive the fine adjustment image data generated by fine adjustment cameras;
process the fine adjustment image data to generate a blood vessel map of an eye of a patient;
select an injection site on the eye of the patient based on the blood vessel map; and
control the fine adjustment actuators to position the needle above the selected injection site for target-confirmed injection of a drug through the needle and into the eye of the patient.

2. The ophthalmic injection system of claim 1, wherein the processor is further configured to:
control the docking assembly to couple the docking assembly to a head of the patient located on the patient support based on the positioning image data; and
control the docking assembly to decouple the docking assembly from the patient based on the positioning image data.

3. The ophthalmic injection system of claim 1, wherein the processor is further configured to:
control the needle extension actuator to extend the needle onto the injection site;
process the fine adjustment image data is processed to confirm that the needle is located on the injection site;
after a needle location on the injection site is confirmed, control the needle extension actuator to extend the needle into the eye of the patient;
control the needle to inject the drug; and
control the needle extension actuator to withdraw the needle from the eye of the patient.

4. The ophthalmic injection system of claim 3, wherein:
the docking assembly further comprises adjustable, high-contrast red green blue (RGB) light sources and adjustable multispectral light sources; and
the fine adjustment image data includes multispectral image data.

5. The ophthalmic injection system of claim 4, wherein the blood vessel map includes at least a portion of a sclera of the eye of the patient.

6. The ophthalmic injection system of claim 5, wherein the blood vessel map includes at least a portion of a retina of the eye of the patient.

7. The ophthalmic injection system of claim 6, wherein the processor is further configured to:
identify a plurality of candidate injection sites based on an injection type and the blood vessel map, each candidate injection site comprising a location and a score; and
select the injection site based on the candidate injection site scores.

8. The ophthalmic injection system of claim 7, wherein:
the injection type comprises an intravitreal injection, a suprachoroidal injection, a subconjunctival injection, or a subretinal injection; and
the candidate injection site locations are based on the injection type.

9. The ophthalmic injection system of claim 8, wherein the processor is further configured to:
further identify three of the plurality of candidate injection sites based on patient preference data acquired from one or more prior injections.

10. The ophthalmic injection system of claim 8, wherein the processor is further configured to execute one or more machine learning (ML) models to process the fine adjustment image data, identify the candidate injection sites, and select the injection site.

11. The ophthalmic injection system of claim 8, wherein the processor is further configured to:
receive an injection site approval command or an injection site disapproval command for the selected injection site;
responsive to the injection site approval command, control the fine adjustment actuators to position the needle above the injection site for target-confirmed injection of the drug through the needle and into the eye of the patient; and
responsive to the injection site disapproval command, not control the fine adjustment actuators to position the needle above the injection site for target-confirmed injection of the drug through the needle and into the eye of the patient.

12. The ophthalmic injection system of claim 11, wherein:
the ophthalmic injection robot further comprises a display and an input device; and
the processor is further configured to:
present, on the display, the blood vessel map and the selected injection site; and
responsive to a user input, generate the injection site approval command or the injection site disapproval command.

13. The ophthalmic injection system of claim 11, further comprising:
a visualization computer coupled to the ophthalmic injection robot over a network, the visualization computer comprising a display, an input device, a memory, and a processor configured to:
receive, over the network, the blood vessel map and the selected injection site from the ophthalmic injection robot;
present, on the display, the blood vessel map and the selected injection site;
responsive to a user input, generate the injection site approval command or the injection site disapproval command; and
send, over the network, the injection site approval command or the injection site disapproval command to the ophthalmic injection robot.

14. The ophthalmic injection system of claim 13, wherein:
the candidate injection sites are located within a predetermined distance from a limbus of the eye; and
the processor of the visualization computer is further configured to:
superpose a guide ring on the blood vessel map at the predetermined distance, the guide ring being centered on a pupil of the eye, and
present, on the display, the guide ring, the blood vessel map, the candidate injection sites, and the selected injection site.

15. The ophthalmic injection system of claim 14, wherein the predetermined distance is mm±1 mm.

16. The ophthalmic injection system of claim 8, wherein:
the injection type is the subretinal injection;
the ophthalmic injection robot further comprises an optical coherence tomography (OCT) sensor configured to generate multidimensional retina image data; and
the processor is further configured to:
determine a retina thickness based on the multidimensional retina image data, and
further identify the plurality of candidate injection sites based on the retina thickness.

17. A processor-based method for performing an ophthalmic injection using an ophthalmic injection robot, comprising:
receiving fine adjustment image data generated by fine adjustment cameras;
processing the fine adjustment image data to generate a blood vessel map of an eye of a patient;
selecting an injection site on the eye of the patient based on the blood vessel map; and
controlling fine adjustment actuators to position a needle above the selected injection site for target-confirmed injection of a drug through the needle and into the eye of the patient.

18. The processor-based method of claim 17, further comprising:
controlling a needle extension actuator to extend the needle onto the injection site;
processing the fine adjustment image data is processed to confirm that the needle is located on the injection site;
after a needle location on the injection site is confirmed, controlling the needle extension actuator to extend the needle into the eye of the patient;
controlling the needle to inject the drug; and
controlling the needle extension actuator to withdraw the needle from the eye of the patient.

19. The processor-based method of claim 18, wherein:
the ophthalmic injection robot comprises:
a frame comprising a patient support and positioning cameras;
a robotic arm coupled to a docking assembly, the docking assembly comprising fine adjustment cameras, and a staging assembly including the fine adjustment actuators, the needle extension actuator, and a needle assembly including the needle,
a control system comprising a memory and a processor, and
one or more adjustable, high-contrast red green blue (RGB) light sources and one or more adjustable multispectral light sources configured to generate multispectral image data; and
the fine adjustment image data includes multispectral image data.

20. The processor-based method of claim 17, further comprising:
identifying a plurality of candidate injection sites based on an injection type and the blood vessel map, each candidate injection site comprising a location and a score; and
selecting the injection site based on the candidate injection site scores.
